(19) United States
(12) Patent Application Publication
(10) Pub. No.: US 2006/0064146 A1
(43) Pub. Date: Mar. 23, 2006

(54) Heating/cooling system for indwelling heat exchange catheter

(76) Inventor: Kenneth A. Collins, Mission Viejo, CA (US)

Correspondence Address: ROGITZ & ASSOCIATES, 750 B STREET, SUITE 3120, SAN DIEGO, CA, 92101, US

(21) Appl. No.: 10/944,544

(22) Filed: Sep. 17, 2004

(51) Int. Cl. A61F 7/00; A61F 7/12

(52) U.S. Cl. 607/105; 607/113

(57) ABSTRACT

A cooling system for an indwelling heat exchange catheter includes a heat exchange bath that is configured to receive a conduit that carries saline to and from the catheter. A heating/cooling fluid is in the bath and exchanges heat with the saline. A pump circulates the patient coolant to and from the catheter. The bath is kept at a minimum temperature and the pump stopped, with the pump being started immediately upon receiving a signal indicating that the patient's temperature is rising above a desired setpoint.

### FIELD OF THE INVENTION

The present invention relates generally to methods and apparatus for exchanging heat with the body of a patient.

### DESCRIPTION OF THE RELATED ART

It has been discovered that the medical outcome for a patient suffering from severe brain trauma or from ischemia caused by stroke or heart attack or cardiac arrest is improved if the patient is cooled below normal body temperature (37° C.). Furthermore, it is also accepted that for such patients; it is important to prevent hyperthermia (fever) even if it is decided not to induce hypothermia. Moreover, in certain applications such as post-CABG surgery, it might be desirable to rewarm a hypothermic patient.

As recognized by the present invention, the above-mentioned advantages in regulating temperature can be realized by cooling or heating the patient's entire body. Moreover, the present invention understands that since many patients already are intubated with central venous catheters for other clinically approved purposes anyway such as drug delivery and blood monitoring, providing a central venous catheter that can also cool or heat the blood requires no additional surgical procedures for those patients. The following U.S. patents, all of which are incorporated herein by reference, disclose various intravascular catheters/systems/methods: U.S. Pat. Nos. 6,749,625, 6,419,643, 6,416,533, 6,409,747, 6,405,080, 6,393,320, 6,368,304, 6,338,727, 6,299,599, 6,290,717, 6,287,326, 6,165,207, 6,149,670, 6,146,411, 6,126,684, 6,306,161, 6,264,679, 6,231,594, 6,149,676, 6,149,673, 6,110,168, 5,989,238, 5,879,329, 5,837,003, 6,383,210, 6,379,378, 6,364,899, 6,325,818, 6,312,452, 6,261,312, 6,254,626, 6,251,130, 6,251,129, 6,245,095, 6,238,428, 6,235,048, 6,231,595, 6,224,624, 6,149,677, 6,096,068, 6,042,559, and U.S. patent application Ser. No. 10/355,776.

Regardless of the particular catheter used, it is clear that heat must be removed from or added to the coolant that flows through the catheter. The present invention critically recognizes that in some contexts, such as when a patient becomes rapidly hyperthermic (“spikes a fever”), it is important to cool the patient with as little delay as possible.

### SUMMARY OF THE INVENTION

A heat exchange system for cooling a patient coolant circulating through a closed loop intravenous catheter includes a cooling receptacle that can be engaged with a portion of a path of the patient coolant outside the catheter. A working fluid is in thermal contact with the cooling receptacle to cool the patient coolant flowing through the path, with a pump circulating patient coolant between the catheter and the path in the cooling receptacle. A heat sink is in thermal contact with the working fluid. According to the present invention, a controller receives a patient temperature signal and controls the heat sink and pump such that the working fluid is maintained at a low temperature and the pump is deenergized when a patient in whom the catheter is intubated is at a target temperature. The low temperature may be at least ten degrees Celsius below normal body temperature, and preferably may be substantially zero degrees Celsius. The controller energizes the pump when the patient's temperature rises above the target temperature such that the patient is immediately cooled.

In non-limiting implementations the patient coolant may be saline and the path may be defined at least in part by a tubing set that is disposable in the cooling receptacle. The target temperature may be in the normothermia range in which case the pump is energized substantially as soon as the signal indicates that a patient is spiking a fever. Or, the target temperature may be in the mild or moderate hypothermia range in which case the pump is energized substantially as soon as the patient temperature drifts up from target temperature.

In another aspect, a patient temperature control system includes an intravascular closed loop catheter through which patient coolant circulates under the influence of a pump. A cooling member holds working fluid in thermal contact with the patient coolant. In accordance with present principles, a processor receives a patient temperature signal and when the signal indicates that the patient is at or below a target temperature, the processor maintains the working fluid substantially at zero degrees Celsius and the pump deenergized. The processor causes the pump to be energized when the patient temperature signal indicates that the patient's temperature is rising above target temperature.

In still another aspect, a method for treating a patient includes advancing a closed loop heat exchange catheter into the vasculature of the patient, and circulating patient coolant through the catheter and past a working fluid using a pump to lower the temperature of the patient to a target temperature. The method then includes deenergizing the pump while maintaining the working fluid at a temperature of no more than ten degrees Celsius. Upon indication of the patient's temperature rising above target temperature, the pump is started.

In still another aspect, a system for treating a patient includes a closed loop heat exchange catheter advanceable into the vasculature of the patient. Means are provided for circulating patient coolant through the catheter and past a working fluid to lower the temperature of the patient to a target temperature. Also, means are provided for deenergizing the pump and for maintaining the working fluid at a temperature of no more than approximately zero degrees Celsius. Additional means, upon indication of the patient's temperature rising above target temperature, starts the pump.

The details of the present invention, both as to its construction and operation, can best be understood in reference to the accompanying drawings, in which like numerals refer to like parts, and which:

### BRIEF DESCRIPTION OF THE DRAWINGS

FIG. 1 is a block diagram of an exemplary patient cooling system; and

FIG. 2 is a flow chart of the present logic.

### DETAILED DESCRIPTION OF THE PREFERRED EMBODIMENT

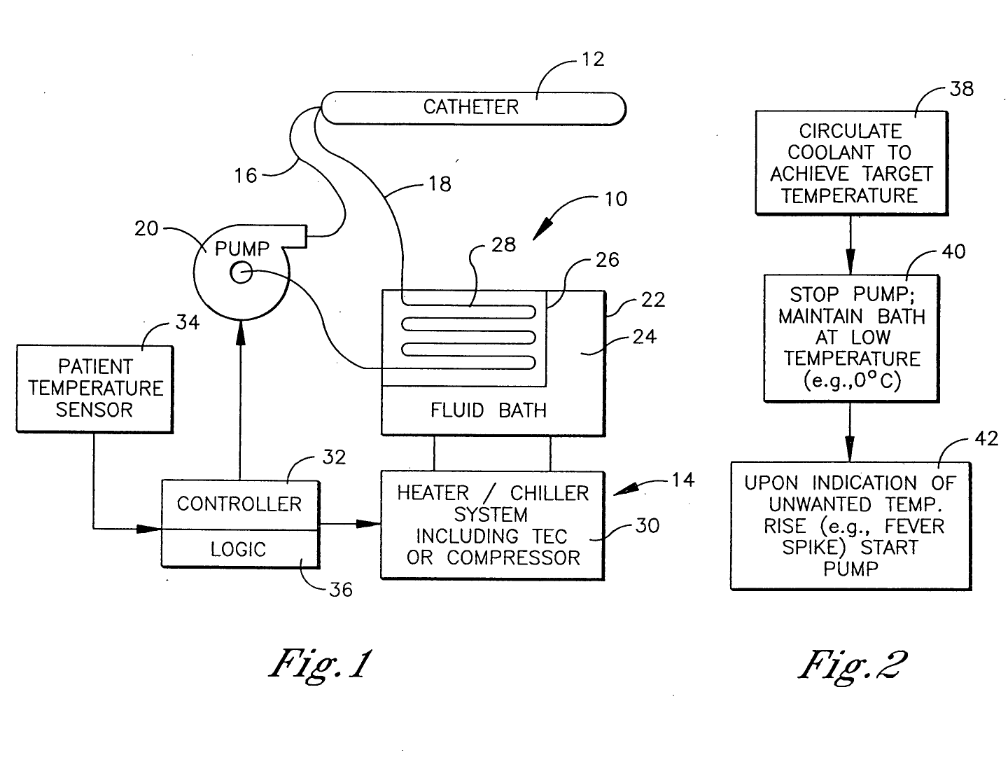

Referring initially to FIG. 1, a patient heat exchange system is shown and generally designated 10. The system 10 includes an indwelling heat exchange catheter 12 that can be inserted into a patient to heat or cool the patient. The catheter 12 may be any of the catheters disclosed in the above-referenced patents or other appropriate closed loop heat exchange catheters.

Coolant such as but not limited to saline is circulated through the catheter 12 in a closed loop to and from a member such as a heat exchange system 14 through coolant supply and return tubes 16, 18 under the influence of a pump 20 (such as but not limited to a gear pump, roller pump, diaphragm pump, or other type of pump) to heat or cool the coolant as desired to warm or cool a patient. The catheter 12 is made of biocompatible material that may be coated with an anti-coagulant substance such as Heperin®. Preferably, the catheter 12 is made of flexible plastic, and on its distal end it may include one or more heat exchange elements such as balloons or fibers or metallic structures.

In any case, the catheter 12 is sized to fit within the patient's bloodstream without blocking blood flow and without allowing coolant to enter the bloodstream. The blood can flow around substantially all of the exposed surface areas of the heat exchange elements disclosed below when the catheter 12 is positioned in the bloodstream and coolant is being circulated through the catheter, to exchange heat with the blood. In a preferred embodiment, the catheter 12 is configured for placement within the venous system, preferably in the superior vena cava or inferior vena cava through the jugular vein or subclavian vein or femoral vein. Less preferably the catheter 12 may be positioned in the arterial system.

Preferred non-limiting uses for the catheter 12 include preventing the onset of fever in patients by keeping the patients normothermic. Other uses include inducing mild or moderate therapeutic hypothermia in patients suffering a cardiac arrest, acute myocardial infarction, stroke, brain trauma, or undergoing aneurysm surgery. The catheter 12 may also be used to rewarm such patients as well as rewarm patients post-surgery, e.g., post-cardiac bypass surgery.

In the particular non-limiting embodiment shown in FIG. 1, the cooling system 14 includes a working fluid bath container 22 in which a working fluid bath 24 such as saline, glycol, a mixture thereof, or other appropriate working fluid is disposed. The container 22 may define a cooling receptacle 26 that can receive a tubing set 28 through which coolant flows as part of the closed coolant path. The tubing set 28 may be implemented as a single length of IV tubing or, as indicated in FIG. 1, the tubing set 28 may include a serpentine-like coolant path in a bag-like cartridge assembly that can be easily engaged and disengaged with the receptacle 26. In any case, it will be appreciated that the working fluid in the bath 24 is in thermal contact with the cooling receptacle 26 and, hence, with the coolant in the tubing set 28 to cool the patient coolant flowing through the path when the patient coolant is warmer than the working fluid.

The cooling system 14 also includes a heat sink 30 that is in thermal contact with the working fluid in the bath 24. The working fluid may be circulated between the heat sink 30 and the bath 24. The heat sink 30 may be a combined heater/chiller system that can include a refrigerant compressor and/or a thermo-electric cooler (TEC) to cool working fluid. Details of various types of non-limiting heat sinks are set forth in selected of the above-referenced U.S. patents.

FIG. 1 shows that a controller 32 receives a patient temperature signal from a temperature sensor 34. In accordance with present principles, the controller 32 accesses a logic module 36 to control the heat sink 30 and pump 20 in accordance with logic set forth further below. The controller 32 may be implemented by any suitable processor. The temperatures sensor 34 may be any suitable temperature sensor such as a thermocouple, resistance temperature detector (RTD), tympanic IR sensor, or other sensor that outputs a signal representative of patient temperature, preferably patient core temperature. The sensor 34 may be placed in the bloodstream of the patient, or in the esophagus, rectum, bladder, or near the ear canal to sense tympanic temperature, in accordance with patient temperature sensing principles known in the art. The logic module 36 may be implemented in electronic storage such as disk or solid state memory and accessed by a processor to execute the present logic.

Now referring to FIG. 2, the logic of the present invention can be seen. Commencing at block 38, with the catheter 12 having been intubated in the vasculature of the patient, coolant is circulated by the pump 20 under control of the controller 32 through the catheter 12 and through the portion of the coolant flow path outside the body that is cooled by the system 14. Coolant is circulated until target temperature is achieved. For fever control applications target temperature is normothermia. For therapeutic hypothermia applications target temperature may be in the mild or moderate hypothermia range.

At block 40, target temperature is reached. The pump 20 is stopped, and the bath 24 is maintained at a low temperature, e.g., zero degrees Celsius. At block 42, when the signal from the temperature sensor 34 indicates that the patient temperature is rising above target temperature, e.g., that the patient is spiking an unwanted fever, the pump 20 is immediately started by the controller 32 such that cold coolant is immediately supplied to the patient, as opposed to having to wait for the bath to cool down from ambient until maximum cooling can be effected.

While the particular HEATING/COOLING SYSTEM FOR INDWELLING HEAT EXCHANGE CATHETER as herein shown and described in detail is fully capable of attaining the above-described aspects of the invention, it is to be understood that it is the presently preferred embodiment of the present invention and thus, is representative of the subject matter which is broadly contemplated by the present invention, that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural and functional equivalents to the elements of the above-described preferred embodiment that are known or later come to be known to those of ordinary skill in the art are expressly incorporated herein by reference and are intended to be encompassed by the present claims. Moreover, it is not necessary for a device or method to address each and every problem sought to be solved by the present invention, for it is to be encompassed by the present claims. Furthermore, no element, component, or method step in the present disclosure is intended to be dedicated to the public regardless of whether the element, component, or method step is, explicitly recited in the claims. No claim element herein is to be construed under the provisions of 35 U.S.C. section 112, sixth paragraph, unless the element is expressly recited using the phrase “means for.”

What is claimed is:

1. A heat exchange system for cooling a patient coolant circulating through a closed loop intravenous catheter, comprising:
a cooling receptacle engageable with a portion of a path of the patient coolant outside the catheter;
a working fluid in thermal contact with the cooling receptacle at least to cool the patient coolant flowing through the path when the patient coolant is warmer than the working fluid;
a pump circulating patient coolant between the catheter and the path in the cooling receptacle;
a heat sink in thermal contact with the working fluid; and
a controller receiving a patient temperature signal and controlling the heat sink and pump such that the working fluid is maintained at a low temperature at least ten degrees Celsius below normal body temperature and the pump is deenergized when a patient in whom the catheter is intubated is at a target temperature, the controller energizing the pump when the patient's temperature rises above the target temperature such that the patient is immediately cooled.

2. The system of claim 1, wherein the low temperature is at least twenty degrees Celsius below normal body temperature.

3. The system of claim 2, wherein the low temperature is no more than zero degrees Celsius.

4. The system of claim 1, wherein the patient coolant is saline and the path is defined at least in part by a tubing set that is disposable in the cooling receptacle.

5. The system of claim 1, wherein the target temperature is in the normothermia range and the pump is energized substantially as soon as the signal indicates that a patient is spiking a fever.

6. The system of claim 1, wherein the target temperature is in the mild or moderate hypothermia range and the pump is energized substantially as soon as the patient temperature drifts up from target temperature.

7. A patient temperature control system, comprising:
an intravascular closed loop catheter through which patient coolant circulates under the influence of a pump;
a cooling member holding working fluid in thermal contact with the patient coolant; and
a processor receiving a patient temperature signal and when the signal indicates that the patient is at or below a target temperature maintaining the working fluid substantially at zero degrees Celsius and the pump deenergized, the processor causing the pump to be energized when the patient temperature signal indicates that the patient's temperature is rising above target temperature.

8. The system of claim 7, wherein the cooling member includes:
a cooling receptacle engageable with a portion of a path of the patient coolant outside the catheter, the working fluid being in thermal contact with the cooling receptacle at least to cool the patient coolant flowing through the path when the patient coolant is warmer than the working fluid; and
a heat sink in thermal contact with the working fluid, the processor controlling the heat sink and pump.

9. The system of claim 8, wherein the patient coolant is saline and the path is defined at least in part by a tubing set that is disposable in the cooling receptacle.

10. The system of claim 7, wherein the target temperature is in the normothermia range and the pump is energized substantially as soon as the signal indicates that a patient is spiking a fever.

11. The system of claim 7, wherein the target temperature is in the mild or moderate hypothermia range and the pump is energized substantially as soon as the patient temperature drifts up from target temperature.

12. A method for treating a patient, comprising:
advancing a closed loop heat exchange catheter into the vasculature of the patient;
circulating patient coolant through the catheter and past a working fluid using a pump to lower the temperature of the patient to a target temperature;
deenergizing the pump while maintaining the working fluid at a temperature of no more than ten degrees Celsius; and
upon indication of the patient's temperature rising above target temperature, starting the pump.

13. A system for treating a patient, comprising:
a closed loop heat exchange catheter advanceable into the vasculature of the patient;
means for circulating patient coolant through the catheter and past a working fluid to lower the temperature of the patient to a target temperature;
means for deenergizing the pump;
means for maintaining the working fluid at a temperature of no more than approximately zero degrees Celsius; and
means for, upon indication of the patient's temperature rising above target temperature, starting the pump.
